(19) United States
(12) Patent Application Publication
(10) Pub. No.: US 2008/0153832 A1
(43) Pub. Date: Jun. 26, 2008

(54) PAN-ALPHA-2 RECEPTOR AGONIST AND RELAXANT COMPOSITIONS FOR TREATING GASTROINTESTINAL MOTILITY DISORDERS

(75) Inventors: Gregory F. Brooks, Irvine, CA (US); Daniel W. Gil, Corona Del Mar, CA (US)

Correspondence Address: ALLERGAN, INC., 2525 DUPONT DRIVE, T2-7H, IRVINE, CA, 92612-1599, US

(73) Assignee: Allergan, Inc.

(21) Appl. No.: 11/954,595

(22) Filed: Dec. 12, 2007

Related U.S. Application Data
(60) Provisional application No. 60/871,704, filed on Dec. 22, 2006.

(51) Int. Cl. A61K 31/498; A61K 31/136; A61K 31/137; A61K 31/138; A61K 31/15; A61K 31/343; A61P 1/00; A61K 31/4164; A61K 31/433; A61K 31/4525; A61K 31/496

(52) U.S. Cl. 514/249; 514/253.04; 514/338; 514/362; 514/392; 514/396; 514/400; 514/401; 514/469; 514/640; 514/647; 514/651; 514/653

(57) ABSTRACT

Disclosed herein is a pharmaceutical composition comprising a relaxant and a pan-alpha-2 receptor agonist. The composition is effective for treating gastrointestinal motility disorders, and methods of treating such disorders using the composition and compounds comprising it are also disclosed.

### CROSS REFERENCE TO RELATED APPLICATIONS

This application is based on, and claims the benefit of, U.S. Provisional Application No. 60/871,704, filed Dec. 22, 2006, and which is incorporated herein by reference.

Disclosed herein is a pharmaceutical composition comprising a relaxant and pan-alpha-2 receptor agonist. The composition is effective for treating gastrointestinal motility disorders, and methods of treating such disorders using the composition and compounds comprising it are also disclosed. Administering pan-alpha-2 receptor agonist together with a relaxant increases the efficacy of these compounds in treating the gastrointestinal motility disorders.

### DETAILED DESCRIPTION OF THE INVENTION

### Disorders of Gastrointestinal Motility

“Gastrointestinal motility” refers to the movement of food through the gastrointestinal tract. A “disorder of gastrointestinal motility” is any abnormality in that process that causes discomfort to a patient. It includes, for example, achalasia, Barrett's syndrome, biliary dyskinesia, Crohn's disease, chronic intestinal pseudo-obstruction, colonic inertia, constipation, cyclic vomiting syndrome, diarrhea, diffuse esophageal spasm, dumping syndrome, dyspepsia, dysphagia, encopresis, fecal incontinence, functional abdominal pain (e.g., chronic proctalgia, epigastric pain syndrome, functional abdominal pain syndrome, proctalgia fugax), functional biliary disorders (e.g., functional biliary SO disorder, functional gallbladder disorder, functional pancreatic SO disorder, functional sphincter of Oddi disorder), functional bowel outlet obstruction, functional dyspepsia disorders (e.g., epigastric pain syndrome, functional dyspepsia, postprandial distress syndrome), functional esophogeal disorders (e.g., functional chest pain of presumed esophogeal origin, functional dysphagia, functional heartburn, globus), functional fecal retention, gastroesophageal reflux disease (GERD), gastroparesis, gastritis, gastropathy, Hirschprung's disease, hypercontractile motility, hypermotility, hypertensive lower esophageal sphincter, hypomotility, intestinal obstruction, irritable bowel syndrome, ischemia, megacolon, non-erosive reflux disease, pancreatitis, pelvic floor dysfunction, short bowel syndrome, small bowel bacterial overgrowth, small bowel intestinal motility disorder, superior mesenteric artery syndrome, ulcerative colitis, and volvulus.

It also includes any symptom produced by disorders of gastrointestinal motility that results in discomfort to a patient, regardless of how one would categorize the disorder that creates the discomfort. Hence, “disorder of gastrointestinal motility” also includes, for example, altered bowel habit (including, for example, change in stool frequency; change in stool form, such as passing hard or loose stools; or change in the manner of passing stool, such as straining, urgency, or feeling or incomplete evacuation), belching, bloating (including a feeling of abdominal distension), blood or mucus in the stool, diarrhea, dyspepsia, dysphagia, flatulence, globus, hoarseness of voice, loss of appetite, nausea, pain in any area or the chest, colon, stomach, or elsewhere in the abdomen, pyrosis (heartburn), regurgitation, sore throat, trapped gas, and uncomfortable fullness after meals.

### Relaxants

Relaxants useful in the method of the invention include antispasmodic agents, antidepressants, barbiturates, tranquilizers, selective serotonin reuptake inhibitors, and 5-HT3 and 5-HT4 serotonin receptor antagonists.

Antispasmodic agents useful in the invention include anticholingeric agents, such as atracurium, atropine (d/l-hyosycamine), benztropine, chlordiazepoxide, clindinium, darifenacin, dicyclomine, doxacurium, flavoxate, ipratropium, mivacurium, oxybutynin, pancuronium, pirenzepine, scopolamine (l-hyoscine), solifenacin, suxamethonium chloride, tiotropium, tolterodine, trimethaphan, tropicamide, tubocurarine, vecuronium, and combinations thereof.

Antidepressants useful in the invention include selective serotonin reuptake inhibitors, such as citalopram, escitalopram, fluoxetine, mirtazapine, paroxetine, sertraline, and combinations of any of the foregoing.

Barbiturates useful in the invention include secobarbital, mephobarital, pentobarbital, phenobarbital, and combinations thereof.

Tranquilizers include benzodiazepines such as diazepam, clonazepam, alprazolam, temazepam, chlordiazepoxide, flunitrazepam, lorazepam, clorazepate, and combinations thereof.

5-HT3 serotonin receptor antagonists include alosetron, dolasetron, granisteron, ondansetron, and palonosetron. 5-HT4 serotonin receptor antagonists include L-Lysine.

### Selective Serotonin Reuptake Inhibitors

Compounds that inhibit serotonin reuptake, called selective serotonin reuptake inhibitors, or SSRIs, are well known. They include citalopram, dapoxetine, escitalopram, fluoxetine, fluvoxamine, paroxetine, sertraline, and trazodone. Any of these compounds (and any other SSRI), including any of their pharmaceutically acceptable salts, and any of their prodrugs, may be used in the compositions and methods of the invention.

Citalopram, also known as nitalapram, is a selective serotonin reuptake inhibitor having the formula

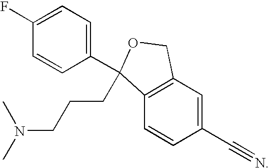

The hydrobromide salt of citalopram is sold in the United States under the brand name Celexa®, and is sold in Europe under the brand names Cipramil and Seropram. Celexa is administered to treat depression at an adult dose of between 40-60 mg/day, beginning with an initial dose of 20 mg/day. Citalopram is described in U.S. Pat. No. 4,136,193, the contents of which are incorporated by reference herein.

Dapoxetine is a selective serotonin reuptake inhibitor having the formula

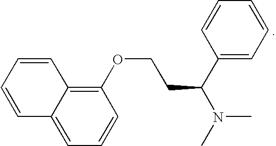

The hydrochloride salt of dapoxetine is currently under review at the U.S. Food and Drug Administration for the treatment of men with premature ejaculation. It is administered at a dose of 60 mg 1-3 hours before the onset of sexual activity. Dapoxetine is short acting, with a time to maximum serum concentration of about 1 hour and an initial half-life of 1.2 hours.

Escitalopram is a selective serotonin reuptake inhibitor having the formula

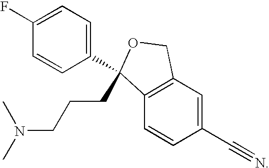

It is the enantiomer of citalopram. The oxalate salt of escitalopram is sold in the United States under the brand name Lexapro®. Escitalopram is administered to treat depression at an adult dose of 10 mg once daily, although may be administered at doses as high as 20 mg/day. Escitalopram is described in U.S. Pat. No. RE 34,712, the contents of which are incorporated by reference herein.

Fluoxetine is a selective serotonin reuptake inhibitor having the formula

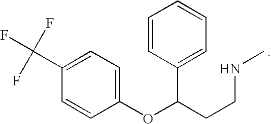

The oxalate salt of fluoxetine is sold in the United States under the brand name Prozac®. Fluoxetine is administered to treat depression at an adult dose of 20-80 mg/day, beginning with an initial dose of 20 mg/day. Fluoxetine is administered to treat depression at a dose of 10-20 mg/day in children, beginning with an initial dose of 10 or 20 mg/day.

Fluvoxamine is a selective serotonin reuptake inhibitor having the formula

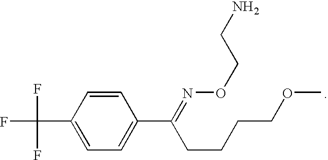

The maleate salt of fluvoxamine is sold in the United States under the brand name Luvox®. Fluvoxamine is administered to treat depression at an adult dose of 100-300 mg/day, beginning with an initial dose of 50 mg, increased in 50-mg increments. It is advised that doses above 100 mg are given in divided doses.

Paroxetine is a selective serotonin reuptake inhibitor having the formula

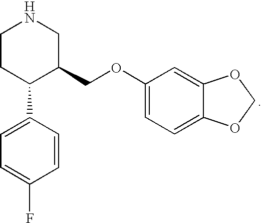

The hydrochloride salt of paroxetine is sold in the United States under the brand name Paxil®. Paroxetine is administered to treat depression at an adult dose of 20-50 mg/day, beginning with an initial dose of 20 mg, increased, if needed, in 10-mg/day increments.

Sertraline is a selective serotonin reuptake inhibitor having the formula

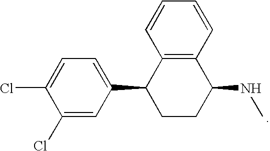

The hydrochloride salt of sertraline is sold in the United States under the brand name Zoloft®. Sertraline is administered to treat depression at an adult dose of 50 mg once daily, but doses as high as 200 mg/day may be used. The initial adult dose is 25 mg once daily.

Trazodone is a selective serotonin reuptake inhibitor having the formula

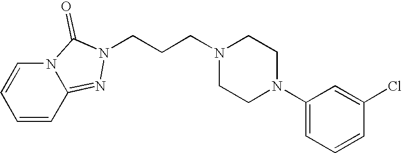

The hydrochloride salt of trazodone is sold in the United States under the brand name Desyrel®. Trazodone is administered to treat depression at an initial adult dose of 150-200 mg daily, divided over two or three doses; the dose is increased in 50 mg increments, as needed to as high as 600 mg daily.

### Pharmaceutically Acceptable Salts

One can use in the compositions and methods of the invention any relaxant as its pharmaceutically acceptable salt.

A “pharmaceutically acceptable salt” is any salt that retains the activity of the parent compound and does not impart any additional deleterious or untoward effects on the subject to which it is administered and in the context in which it is administered compared to the parent compound. A pharmaceutically acceptable salt also refers to any salt which may form in vivo as a result of administration of an acid, another salt, or a prodrug which is converted into an acid or salt.

Pharmaceutically acceptable salts of acidic functional groups may be derived from organic or inorganic bases. The salt may comprise a mono or polyvalent ion. Of particular interest are the inorganic ions lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

### Prodrugs

One can use in the compositions and methods of the invention a prodrug of any relaxant.

A “prodrug” is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, a prodrug is inactive or less active than the therapeutically active compound to which it is converted. Ester prodrugs of the compounds disclosed herein are specifically contemplated. An ester may be derived from a carboxylic acid of C1 (i.e., the terminal carboxylic acid of a natural prostaglandin), or an ester may be derived from a carboxylic acid functional group on another part of the molecule, such as on a phenyl ring. While not intending to be limiting, an ester may be an alkyl ester, an aryl ester, or a heteroaryl ester. The term alkyl has the meaning generally understood by those skilled in the art and refers to linear, branched, or cyclic alkyl moieties. C₁₋₆ alkyl esters are particularly useful, where alkyl part of the ester has from 1 to 6 carbon atoms and includes, but is not limited to, methyl, ethyl, propyl, isopropyl, n-butyl, sec-butyl, iso-butyl, t-butyl, pentyl isomers, hexyl isomers, cyclopropyl, cyclobutyl, cyclopentyl, cyclohexyl, and combinations thereof having from 1-6 carbon atoms, etc.

The relaxants and pan-alpha-2 receptor agonists of the invention may be either synthetically produced, or may be produced within the body after administration of a prodrug. Hence, “relaxant” and “pan-alpha-2 receptor agonist” encompass compounds produced by a manufacturing process and those compounds formed in vivo only when another drug administered.

### Isomers and Racemates

One can use in the compositions and methods of the invention an enantiomer, stereoisomer, or other isomer of any relaxant.

### Pan-Alpha-2 Receptor Agonists

Pan-alpha-2 adrenergic receptor agonists are those compounds that activate the three alpha-2 adrenergic receptor subtypes. A compound is a “pan-alpha-2 receptor agonist” if it has greater than 25% efficacy relative to brimonidine at each of the alpha-2A, alpha-2B, and alpha-2C adrenergic receptors; as long as the agonist meets this definition, it encompasses other receptors, as well (e.g., the agonist can be a pan-alpha-1 adrenergic receptor agonist as well as a pan-alpha-2 receptor agonist). A variety of pan-alpha-2 receptor agonists are known in the art, such as brimonidine, clonidine, dexmedetomidine, mivazerol, norepinephrine, oxymetazoline, and tizanidine. A pan-alpha-2 receptor agonist has, at a minimum, greater than 25% efficacy relative to brimonidine at each of the alpha-2A, alpha-2B and alpha-2C receptors; in particular embodiments, a method of the invention is practiced with a pan-alpha-2 receptor agonist having greater than 30%, 40%, 50%, 60%, 70%, 80%, 90%, 100% or 200% efficacy relative to brimonidine at the alpha-2A, alpha-2B and alpha-2C adrenergic receptors. It is understood that the efficacy of a pan-alpha-2 receptor agonist can be different at the various alpha-2 receptors; as an example, a pan-alpha-2 receptor agonist can have greater than 25% efficacy at the alpha-2A receptor, greater than 80% efficacy at the alpha-2B receptor and greater than 40% efficacy at the alpha-2C receptor

Efficacy, also known as intrinsic activity, is a measure of maximal receptor activation achieved by a compound and can be determined using any accepted assay of alpha-adrenergic receptor activation, such as a cAMP or Receptor Selection and Amplification Technology (RSAT). Efficacy is represented as a ratio or percentage of the maximal effect of the drug to the maximal effect of a standard agonist for each receptor subtype. Brimonidine, itself a pan-alpha-2 receptor agonist (it is has 100% of the efficacy of brimonidine at the alpha-2A, alpha-2B, and alpha-2C adrenergic receptors), is used as the standard agonist for the alpha-2 receptors.

Agonist activity can be characterized using any of a variety of routine assays, including, for example, Receptor Selection and Amplification Technology (RSAT) assays (Messier et al., Pharmacol. Toxicol. 76:308-11 (1995); cyclic AMP assays (Shimizu et al., J. Neurochem. 16:1609-1619 (1969)); and cytosensor microphysiometry assays (Neve et al., J. Biol. Chem. 267:25748-25753 (1992)). Such assays generally are performed using cells that naturally express only a single alpha-adrenergic receptor subtype, or using transfected cells expressing a single recombinant alpha-adrenergic receptor subtype. The adrenergic receptor can be a human receptor or homolog of a human receptor having a similar pharmacology.

The RSAT assay measures receptor-mediated loss of contact inhibition resulting in selective proliferation of receptor-containing cells in a mixed population of confluent cells. The increase in cell number is assessed with an appropriate detectable marker gene such as beta-galactosidase, if desired, in a high throughput or ultra high throughput assay format. Receptors that activate the G protein, Gq, elicit the proliferative response. Alpha-adrenergic receptors, which normally couple to Gi, activate the RSAT response when coexpressed with a hybrid Gq protein containing a Gi receptor recognition domain, designated Gq/i5. Conklin et al., Nature 363:274-6 (1993)).

As an example, an RSAT assay can be performed essentially as follows. NIH-3T3 cells are plated at a density of 2×10⁶ cells in 15 cm dishes and maintained in Dulbecco's modified Eagle's medium supplemented with 10% calf serum. One day later, cells are cotransfected by calcium phosphate precipitation with mammalian expression plasmids encoding p-SV-β-galactosidase (5-10 μg), receptor (1-2 μg) and G protein (1-2-μg). Carrier DNA, for example 40 μg salmon sperm DNA, also can be included to increase transfection efficiency. Fresh media is added on the following day; one to two days later, cells are harvested and frozen in 50 assay aliquots. Transfected cells are thawed, and 100 μl of cells added to 100 μl aliquots of compound to be tested, with various concentrations assayed in triplicate, for example, in 96-well plates. Incubation continues for 72 to 96 hours at 37° C. After washing with phosphate-buffered saline, β-galactosidase activity is determined by adding 200 μl of chromogenic substrate (3.5 mM O-nitrophenyl-β-D-galactopyranoside/0.5% NP-40 in phosphate buffered saline), incubating overnight at 30° C., and measuring optical density at 420 nm. The absorbancy is a measure of enzyme activity, which depends on cell number and reflects receptor-mediated cell proliferation. The EC₅₀ and maximal effect (i.e., efficacy) of each drug at each receptor is determined.

Exemplary pan-alpha-2-receptor agonists include the compounds below in Table 1:

TABLE 1
Pan-alpha-2 receptor agonists

| COMPOUND | STRUCTURE |
|---|---|
| 1 | 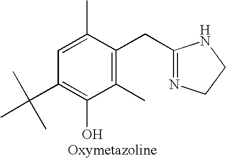 |
| 2 | 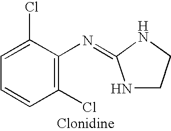 |
| 3 | 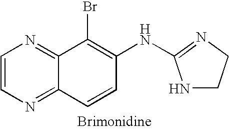 |
| 4 | 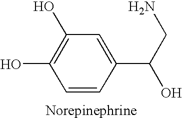 |
| 5 | 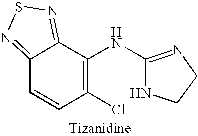 |
| 6 | 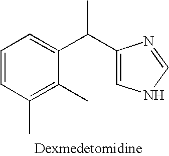 |
| 7 | 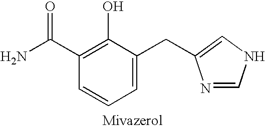 |

One can use in the methods and compositions of the invention any pharmaceutically acceptable salt, prodrug, isomer, and racemate (as those terms are defined in the preceding sections) of any pan-alpha-2 receptor agonist.

### Pharmaceutical Compositions

Pharmaceutical compositions of the invention comprise one or more of a relaxant and one or more of a pan-alpha-2 receptor agonist.

### Excipients and Dosage Forms

Those skilled in the art will readily understand that for administering pharmaceutical compositions of the invention relaxants and pan-alpha-2 receptor agonists can be admixed with pharmaceutically acceptable excipient which are well known in the art.

A pharmaceutical composition to be administered systemically may be confected as a powder, pill, tablet or the like, or as a solution, emulsion, suspension, aerosol, syrup or elixir suitable for oral or parenteral administration or inhalation.

For solid dosage forms or medicaments, non-toxic solid carriers include, but are not limited to, pharmaceutical grades of mannitol, lactose, starch, magnesium stearate, sodium saccharin, the polyalkylene glycols, talcum, cellulose, glucose, sucrose and magnesium carbonate. The solid dosage forms may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in U.S. Pat. No. 4,256,108, No. 4,166,452, and No. 4,265,874 to form osmotic therapeutic tablets for control release. Liquid pharmaceutically administrable dosage forms can, for example, comprise a solution or suspension of one or more of the presently useful compounds and optional pharmaceutical adjutants in a carrier, such as for example, water, saline, aqueous dextrose, glycerol, ethanol and the like, to thereby form a solution or suspension. If desired, the pharmaceutical composition to be administered may also contain minor amounts of nontoxic auxiliary substances such as wetting or emulsifying agents, pH buffering agents and the like. Typical examples of such auxiliary agents are sodium acetate, sorbitan monolaurate, triethanolamine, sodium acetate, triethanolamine oleate, etc. Actual methods of preparing such dosage forms are known, or will be apparent, to those skilled in this art; for example, see Remington's Pharmaceutical Sciences, Mack Publishing Company, Easton, Pa., 16th Edition, 1980. The composition of the formulation to be administered, in any event, contains a quantity of one or more of the presently useful compounds in an amount effective to provide the desired therapeutic effect.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol and the like. In addition, if desired, the injectable pharmaceutical compositions to be administered may also contain minor amounts of non-toxic auxiliary substances such as wetting or emulsifying agents, pH buffering agents and the like.

### Methods of Treatment

The pharmaceutical compositions of the invention may be used to treat motility disorders. To “treat,” as used here, means to deal with medically. It includes administering agents of the invention to prevent the onset of a condition, ameliorate its symptoms, address its cause, or to prevent its reoccurrence. All these things fall within the meaning of “treating.”

One can treat, according to the method of the invention, motility disorders or their symptoms by administering to a patient a combination of one or more of a relaxant and one or more of a pan-alpha-2 receptor agonist. The foregoing agents may be administered together, but one can also administer these compounds separately, administering one immediately after the other, or administering one within a short interval after the other (e.g., 5-15 minutes, or 15-30 minutes, or 30 minutes-1 hour), or administering one within a longer interval after the other (e.g., 1-2 hours, 2-4 hours, 4-6 hours, 6-12 hours, or 12-24 hours). One can also administer one compound more frequently than another, administering, for example, a relaxant one or more times daily and a pan-alpha-2 receptor agonist two or more times daily (or vice versa).

The relaxants and pan-alpha-2 receptor agonists of the invention may be administered in a single formulation (e.g., a single pill or injection), or may be administered separately, each in its own formulation (e.g., a proton pump inhibitor orally once daily and a pan-alpha-2 receptor agonist twice daily via injection).

A patient may be administered the usual course of relaxant and the usual course of alpha-2 agonist, but a patient may also receive a reduced course of one or the other therapy or of both therapies (that is, a patient may take a lower dose than is usually prescribed or may take it for a shorter duration).

An “effective dose,” means a dose which reduces discomfort in a patient to tolerable levels.

### Dose

Pharmaceutical compositions of the invention may be formulated such that a patient receives a dose of an antrelaxant that is usually effective, when administered separately, to treat a motility disorder, and a dose of a pan-alpha-2 receptor agonist that is usually effective, when administered separately, to treat a motility disorder. But the pharmaceutical compositions of the invention may also be formulated such that doses of each compound may be those that are ineffective or minimally effective when the compounds are administered alone. This allows one to administer to a patient a formulation of the invention that is as effective as a larger dose of a relaxant or pan-alpha-2 receptor agonist when administered alone, but less likely to lead to side effects. This does not mean, however, that formulations of the invention comprise relaxants and pan-alpha-2 receptor agonists in only such doses which are, when administered alone, minimally effective: a patient with severe discomfort may require a high dose of either component of the formulation, but is still likely to experience enhanced symptom relief (as compared to the relief the patient would experience were he administered a high dose of either component of the invention alone).

The precise dose and frequency of administration depends on the severity and nature of the patient's condition, on the manner of administration, on the potency and pharmacodynamics of the particular compound employed, and on the judgment of the prescribing physician. Determining dose is a routine matter that is well within the capability of someone of ordinary skill in the art. The usual effective dose of relaxants are set forth in the tables below as a guide.

TABLE 2
usual effective doses of some common antispasmodics

| ANTISPASMODIC AGENT | ADULT DOSE |
|---|---|
| Dicyclomine | 20 mg-40 mg four times a day orally |
| | 20 mg four times a day intramuscularly |
| Benztropine | 0.5 mg-6 mg per day |

TABLE 3
usual effective doses of some common antidepressants

| ANTIDEPRESSANT | ADULT DOSE |
|---|---|
| Citalopram | 20 mg-40 mg once daily |
| Escitalopram | 10 mg once daily |
| Paroxetine | 25 mg-62.5 mg once daily |
| Fluoxetine | 20-40 mg once or twice daily |
| Sertraline | 25-50 mg once daily |

TABLE 4
usual effective doses of some common barbiturates

| BARBITURATE | ADULT DOSE |
|---|---|
| Mephobarbital | 400 mg-600 mg once daily |
| Pentobarbital | 150 to 200 mg as a single IM injection |
| | 200-500 mg intravenously at 50 mg/min |
| Mephobarbital and | 50 mg to 100 mg of phenobarbital and |
| Phenobarbital | 200 mg to 300 mg of mephobarbital |

TABLE 5
usual effective doses of some common tranquilizers

| TRANQUILIZER | ADULT DOSE |
|---|---|
| Chlordiazepoxide | 5 mg-25 mg, 3 or 4 times daily |
| Alprazolam | 0.25 mg-1.33 mg three times daily |
| Clorazepate | 30 mg-60 mg once daily |
| Diazepam | 2 mg-10 mg, 2-4 times daily |

TABLE 6
usual effective doses of some common 5-HT3 receptor antagonists

| 5-HT3 RECEPTOR | |
|---|---|
| ANTAGONIST | ADULT DOSE |
| Alosetron | 0.5 mg twice daily |
| Dolasetron | 1.8 mg/kg |
| Ondansetron | 24 mg, divided over three |
| | doses in a single setting |
| Palonosetron | 0.25 mg |

What is claimed is:

1. A pharmaceutical composition comprising a pan-alpha-2 receptor agonist and a relaxant selected from the group consisting of an antispasmodic agent, a barbiturate, a tranquilizer, a selective serotonin reuptake inhibitor, and a 5-HT3 receptor antagonist.

2. A method of treating a gastrointestinal motility disorder, the method comprising the step of administering to a patient in need of such treatment one or more of a relaxant and one or more of a pan-alpha-2 receptor agonist.

3. The method of claim 2, wherein the gastrointestinal motility disorder is selected from the group consisting of achalasia, Barrett's syndrome, biliary dyskinesia, Crohn's disease, chronic intestinal pseudo-obstruction, colonic inertia, constipation, cyclic vomiting syndrome, diarrhea, diffuse esophageal spasm, dumping syndrome, dyspepsia, dysphagia, encopresis, fecal incontinence, functional abdominal pain (e.g., chronic proctalgia, epigastric pain syndrome, functional abdominal pain syndrome, proctalgia fugax), functional biliary disorders (e.g., functional biliary SO disorder, functional gallbladder disorder, functional pancreatic SO disorder, functional sphincter of Oddi disorder), functional bowel outlet obstruction, functional dyspepsia disorders (e.g., epigastric pain syndrome, functional dyspepsia, postprandial distress syndrome), functional esophogeal disorders (e.g., functional chest pain of presumed esophogeal origin, functional dysphagia, functional heartburn, globus), functional fecal retention, gastroesophageal reflux disease (GERD), gastroparesis, gastritis, gastropathy, Hirschprung's disease, hypercontractile motility, hypermotility, hypertensive lower esophageal sphincter, hypomotility, intestinal obstruction, irritable bowel syndrome, ischemia, megacolon, non-erosive reflux disease, pancreatitis, pelvic floor dysfunction, short bowel syndrome, small bowel bacterial overgrowth, small bowel intestinal motility disorder, superior mesenteric artery syndrome, ulcerative colitis, and volvulus.

4. The method of claim 2, wherein the gastrointestinal motility disorder is selected from the group consisting of altered bowel habit, belching, bloating, blood or mucus in the stool, diarrhea, dyspepsia, dysphagia, flatulence, globus, hoarseness of voice, loss of appetite, nausea, pain in the chest, pain in the colon, pain in the abdomen, pyrosis, regurgitation, sore throat, trapped gas, and uncomfortable fullness after meals.

5. The method of claim 2, wherein the relaxant is selected from the group consisting of an antispasmodic agent, an antidepressant, a barbiturate, a tranquilizer, a selective serotonin reuptake inhibitor, and a 5-HT3 receptor antagonist.

6. The method of any one of claims 2-5, wherein the relaxant and the pan-alpha-2 receptor agonist are administered in a single formulation.

7. The method of any one of claims 2-5, wherein a first formulation comprising the relaxant and a second formulation comprising the pan-alpha-2 receptor agonist are administered at the same time.

8. The method of any one of claims 2-5, wherein a first formulation comprising the relaxant and a second formulation comprising the pan-alpha-2 receptor agonist are administered at different times.

9. The method of any one of claims 2-5, wherein a first formulation comprising the relaxant is administered once daily and a second formulation comprising the pan-alpha-2 receptor agonist is administered once daily.
